(19) United States
(12) Patent Application Publication
(10) Pub. No.: US 2021/0298646 A1
(43) Pub. Date: Sep. 30, 2021

(54) ORTHOSTATIC REPORTING ASSEMBLY, ORTHOSTATIC REPORTING SYSTEM, AND RELATED METHOD OF USE

(71) Applicants: Michael Pennington, Bella Vista, AR (US); Zion Ong, Saratoga Springs, UT (US)

(72) Inventors: Michael Pennington, Bella Vista, AR (US); Zion Ong, Saratoga Springs, UT (US)

(21) Appl. No.: 17/347,582

(22) Filed: Jun. 14, 2021

Related U.S. Application Data
(63) Continuation-in-part of application No. 17/220,919, filed on Apr. 1, 2021.
Application No. 17/220,919 is a continuation-in-part of application No. 17/151,656, filed on Jan. 18, 2021.
(60) Provisional application No. 62/969,239, filed on Feb. 3, 2020.
(60) Provisional application No. 62/962,933, filed on Jan. 18, 2020.

(51) Int. Cl. A61B 5/11; A61B 5/00

(52) U.S. Cl. CPC A61B 5/1127; A61B 5/1116; A61B 5/6823; A61B 2090/3945; A61B 5/1121; A61B 5/486; A61B 5/6831

(57) ABSTRACT

An illuminable orthostatic reporting apparatus may include a laser pointer disposed within an adjustable housing mount assembly that may be coupled to a harness assembly; the reporting apparatus may be used with a target; the target may be an electronic target configured to report whether a user wearing the reporting apparatus is maintaining a laser beam, emitted from the laser pointer, in an aligned position with the target.

### CROSS-REFERENCE TO RELATED APPLICATIONS

This application incorporates by reference and claims the benefit of the non-provisional U.S. Ser. No. 17/220,919 filed on Apr. 1, 202 which claims the benefit and incorporates by reference U.S. Ser. No. 17/151,656 filed on Jan. 18, 2021, which claims the benefit and incorporates by reference two provisional applications: U.S. 62/969,239 filed on Feb. 3, 2020 and U.S. 62/962,933 filed on Jan. 18, 2020.

### BACKGROUND OF THE INVENTION

Posture is important to health, self-confidence, and well-being.

Various patents and articles have disclosed devices for posture: see https://www.ncbi.nlm.nih.gov/pmc/articles/PMC4201483/ and U.S. Pat. No. 9,406,211.

Current medical apparatus for posture training have limitations. For example, braces provide passive support but not strengthening support. Some other types of medical apparatus give feedback when a user has poor posture but does not significantly strengthen or correct posture issues. Other postural apparatus provide a vibration feedback when the user is not in proper posture, but these apparatus do not provide significant strengthening to correct the posture.

Some researchers have studied participants carrying a tray that is attached to a laser pointer; see https://www.ncbi.nlm.nih.gov/pmc/articles/PMC4201483/. Others have created devices; see U.S. Pat. No. 9,406,211. But a need still exists for a safe, non-intrusive device for assisting users who want to improve their posture.

In summary, current medical apparatus for improving posture have notable shortcomings: a) braces that do not provide a passive strengthening support, and b) medical apparatus that give feedback of poor posture but do not significantly strengthen or correct posture issues.

### BRIEF SUMMARY OF THE INVENTION

The present invention is in the technical field of physical therapy or health-care apparatus for improving posture. More particularly, the present invention is in the technical field of muscular rehabilitation of the postural musculature. Although the present invention is in the technical field of physical therapy, the present invention may be used in multiple fields, such as the medical field, sports training, or life coaching. More particularly, the present invention is also in the technical field of rehabilitation and may be used to train an individual to improve their posture through training. The present invention is in the technical field of muscular rehabilitation of the postural musculature.

The disclosure generally relates to an orthostatic reporting apparatus for prompting a user of the apparatus to alter the user's posture. In some embodiments, an illuminable orthostatic reporting apparatus, includes an orthostatic reporting apparatus coupled to a laser pointer disposed within a portion of an adjustable housing assembly and the orthostatic reporting apparatus may be releasably coupled to a chest mount harness assembly. A system for using the orthostatic reporting apparatus is disclosed: an orthostatic reporting apparatus disposed upon a user and one or more targets mounted on a fixed surface and a user, b) an illuminable apparatus assembly including a laser, c) a system that includes an illuminable apparatus assembly and a target, and d) apparatus, laser, and target to prompt a user to alter posture.

An illuminable orthostatic reporting apparatus is described, which may include an orthostatic reporting assembly coupled to a laser pointer. The illuminable orthostatic reporting apparatus may include a chest mount harness assembly, an adjustable housing assembly coupled to the chest mount harness assembly, a laser pointer, and a target. The chest mount harness assembly may be used to releasably couple the orthostatic reporting assembly to a user. The adjustable housing assembly may be physically connected to the securing system such that the adjustable housing assembly may be releasably coupled or in contact with a sternum. The laser pointer may be mechanically connected to the adjustable housing assembly and may project a point or beam of light. The target may be used to provide a point where the point of light projected by the laser is to be projected.

A nonlimiting method for improving posture is described. The method includes securing an apparatus, for altering posture, to a person; positioning the person a distance from the target of the apparatus for altering posture; maneuvering the adjustable housing assembly such that the laser is projected on the target of the apparatus for altering posture when the person has the desired posture; and the person may stand in place, stand on one foot, take steps, walk in place or walk while striving to maintain the point of light at a consistent place on the target of the apparatus to consciously and continuously alter one's own posture.

The illuminable orthostatic reporting system may also include a forefoot pressure monitor, a heel pressure monitor, a chest angle sensor, a chin angle sensor, or a head crown angle sensor which could be attached via various straps and may interact with other apparatus and operating systems wirelessly.

The first embodiment is an illuminable orthostatic reporting apparatus comprising: an orthostatic reporting assembly comprising an adjustable housing assembly, and a chest mount harness assembly coupled to the adjustable housing assembly; and a pivotable laser pointer disposed within a laser pointer housing of the adjustable housing assembly, wherein the adjustable housing assembly is rotatable around a horizontal axis and rotatable along a vertical axis and z axis, the pivotable laser pointer coupled to interact with the assembly.

The illuminable orthostatic reporting apparatus as of the first embodiment, wherein the adjustable housing assembly is rotatable around vertical axis and z axis by at least 10 degrees.

The illuminable orthostatic reporting apparatus as of the first embodiment, wherein the adjustable housing assembly of the illuminable orthostatic reporting apparatus further comprises a pivotable pointer housing assembly comprising a laser pointer housing (710).

The illuminable orthostatic reporting apparatus as of the first embodiment, wherein the pivotable pointer housing of the pivotable pointer housing assembly of the adjustable housing assembly further comprises a threaded hole coupled to a fastener and an articulating clamp assembly, the articulating clamp assembly further comprising: a plurality of clamp flanges configured to couple together by a clamp assembly fastener, which may be a bolt insertable through a first aperture of the first clamp flange 304c and a second aperture of the second clamp flange 304d.

The illuminable orthostatic reporting apparatus as of the first embodiment, wherein the fastener coupled to the threaded hole (3000) and coupled to the articulating clamp assembly is a ball head fastener comprising a ball head portion and a threaded bolt portion, wherein the threaded bolt portion is mateably coupled to the threaded hole of the laser pointer housing (710) and the ball head portion is rotateably disposed between a third aperture (3100) of the first clamp flange 304c and a fourth aperture 3102 of the second clamp flange 304d; wherein the chest mount harness assembly (130) comprises a main plate (202), the main plate (202) comprising a fastening connector (218) of the main plate (202); wherein a hooked mounting adaptor (302) releasably coupled to the fastening connector (218) and to the articulating clamp assembly (304); wherein the hooked mount adaptor (302) is configured to receive the first clamp.

The illuminable orthostatic reporting apparatus (101) of the first embodiment wherein the pivotable laser pointer housing assembly (700) comprises a laser pointer fastened to the laser pointer housing further comprising at least one fastener (712) that holds the laser pointer in place inside the laser pointer housing.

The illuminable orthostatic reporting apparatus (101) of the first embodiment wherein the pivotable laser pointer housing assembly (700) comprises a laser pointing housing further comprising a fastener ball joint (1100) fastened to the laser pointer housing by a nut (720) and a washer (740a).

The illuminable orthostatic reporting apparatus (101) of the first embodiment wherein articulating clamp assembly (304) comprises a clamp assembly fastener (304e) and a fastener joint connector bolt (1220) that helps keeps level and stable the pivotable laser pointer housing assembly (700).

The illuminable orthostatic reporting apparatus (101) of the first embodiment wherein the hooked mounting assembly comprises a knob covered fastener (302d) and knob covered fastener nut (302e) that fastens the hooked mounting assembly to the articulating clamp assembly.

The second embodiment is an illuminable orthostatic reporting system 1500 comprising: a first target 600a, the first target having a bullseye; and a second target (600b) having a bullseye, the second target being positioned above the first target.

The illuminable orthostatic reporting system of the second embodiment, wherein the first target comprises a light sensor positioned at a center of the bullseye of the first target, the light sensor facing the user, the light sensor being operably coupled to a system comprising: at least one of a sound emitting device (620), a light-emitting device (640), and a display device (680); a processor; and a non-transitory memory storage (660) storing instructions that when executed by the processor cause the processor to perform operations, the operations comprising: a receiving during a first time period, via the light sensor, light intensity data corresponding to an amount of light reflected from the bullseye of the second target; a determination of a length value of a subset time period of the first time period for which the light intensity data corresponding to the amount of light reflected from the bullseye of the second target exceeds an intensity threshold; a calculation of a percentage score by dividing the length value of the subset time period by the first time period; and performing at least one action selected and reporting the percentage score to a display device.

The illuminable orthostatic reporting system of the second embodiment further comprising a system comprising: a processor; a light sensor communicatively coupled to the processor; and a non-transitory storage memory storing instructions that when executed by the processor cause the processor to perform operations, the operations comprising: a receiving during a first time period, via the light sensor, light intensity data corresponding to an amount of light reflected from the bullseye of the second target; a determination of a length value of a subset time period of the first time period for which the light intensity data corresponding to the amount of light reflected from the bullseye of the second target exceeds an intensity threshold; a calculation of a percentage score by dividing the length value of the subset time period by the first time period; and a reporting of the percentage score to a display device.

The illuminable orthostatic reporting system of the second embodiment further comprising a system comprising: a processor; a light sensor communicatively coupled to a processor and coupled to a light sensor that is not physically coupled to the target; the non-transitory storage medium storing a set of instructions configured to instruct the processor to provide biofeedback to the user and display on the display device.

The third embodiment is a method for a user to hold a desirable posture, the method comprising: providing a target having a bullseye; securing an illuminable orthostatic reporting apparatus, comprising an adjustable housing assembly (400) and a laser pointer, to the user; maneuvering the user to a distance from the target; and adjusting the adjustable housing assembly (400) such that a laser beam, having an angle with respect to the horizontal plane, being emitted from the laser pointer is projected onto the bullseye of the target simultaneously with the user having the desirable posture.

The method of the third embodiment wherein the user maintains the desirable posture while simultaneously performing an exercise and maintaining the adjustable housing assembly at a substantially constant angle so as to cause the laser beam to constantly project, for at least 0.1 seconds, onto the bullseye of the target.

The fourth embodiment is an orthostatic reporting system comprising: a forefoot pressure monitor (4040) wirelessly connectable to a system comprising a non-transitory medium and processor; and a heel pressure monitor (4030) wirelessly connectable to a system comprising a non-transitory medium and processor.

The fifth embodiment is an orthostatic reporting system comprising: a chest angle sensor (4020) coupled to the orthostatic reporting assembly (100); a chin angle sensor (4010) which may be held to the chin by straps around the ears (312); and a head crown angle sensor (4000).

The sixth embodiment is an orthostatic reporting system comprising: an angle capturing wireless connection accessible non-transitory storage medium connected camera configured to monitor body angle sensors and transmit findings to a non-transitory storage medium.

The system of the sixth embodiment, wherein the wireless connection accessible system comprises one or more of: a prompt sound responsive biometric feedback; and a data collection and processing response feature.

The system of the sixth embodiment, wherein the user reacts to biometric feedback while performing the method of the third embodiment and thereby creating data for the system of the fifth embodiment to process.

### BRIEF DESCRIPTION OF THE SEVERAL VIEWS OF THE DRAWING

FIG. 1(a) is an illustration of a first head position relative to a first shoulder position in a first posture position. See U.S. Pat. No. 9,406,211.

FIG. 1(b) is an illustration of a second head position relative to a second shoulder position in a head forward position, which is not a correct posture. See U.S. Pat. No. 9,406,211.

FIG. 1(c) is an illustration of a third head position relative to a third shoulder position in a head forward and tilted position, which is also not a correct posture. See U.S. Pat. No. 9,406,211.

FIG. 2 depicts an embodiment of a biofeedback system.

FIG. 3 depicts an embodiment of a laser pointer as seen from a top view.

FIG. 4 depicts an embodiment of a holder coupled via a fastener ball joint fastener to a clamp assembly.

FIG. 5 depicts an embodiment of a target.

FIG. 6 depicts a back view of an embodiment of an illuminable orthostatic reporting apparatus showing a chest mount harness assembly.

FIG. 7 depicts an embodiment of an illuminable orthostatic reporting apparatus.

FIG. 8 depicts an embodiment of an orthostatic reporting assembly.

FIG. 9 depicts a side view of an illuminable orthostatic reporting apparatus, as seen from the right side of a user.

FIG. 10 depicts a close-up side view of the illuminable orthostatic reporting apparatus of FIG. 9, as seen from a left side.

FIG. 11 depicts a top view of an embodiment of the illuminable orthostatic reporting apparatus.

FIG. 12 depicts a bottom view of an embodiment of the illuminable orthostatic reporting apparatus.

FIG. 13 depicts a perspective view of an embodiment of the illuminable orthostatic reporting apparatus.

FIG. 14 depicts an exploded, back-side view of the adjustable housing assembly.

FIG. 15 depicts a back-side view of the adjustable housing assembly with a pointer when mounted to the chest mount harness assembly.

FIG. 16 depicts a front-toppish view of a hooked mounting adaptor 302; the hooked mounting adaptor (302) may couple to chest plate mount (204), shown in FIG. 19, via quick-release clasp (3020) of the hooked mounting adaptor 302, the quick-release clasp (3020) may include a first flexible side flange (302a), a second flexible side flange (302b), and guide slot (302c). As shown in FIG. 19, the central guide flange (202c) may couple with the guide slot (302c) of the hooked mounting adaptor (302), causing the hooked mounting adaptor (302) to be releasably coupled with the fastening connector (218) of the main plate 202. first flexible side flange (302a) may be compressed by frictional contact with the first flange (202a) shown in FIG. 19, and second flexible side flange (302b) may also be compressed by frictional contact with the second flange (202b) shown in FIG. 19 upon insertion of the hooked mounting adaptor into the clip-in mechanism. Main plate (202) may be coupled to a user's chest. Once fully inserted, the plurality of flexible side flanges (302 a, 302bb) may then extend pass the inferior ends of the first flange (202a) and the second flange (202b) of the fastening connector 218 of the main plate 202, thereby releasably coupling the hooked mounting adaptor (302) to the main plate (202).

FIG. 17 depicts a back facing view of the adjustable housing assembly.

FIG. 18 depicts a bottom view facing up of an illuminated orthostatic reporting apparatus.

FIG. 19 shows an embodiment of a portion of chest mount harness assembly showing a metal plate with harness straps.

FIG. 20 shows an embodiment of a laser pointer, having a charging port, coupled to a charging cable, such as a USB cable.

FIG. 21a shows an embodiment of a laser pointer housing from a front-bottom perspective.

FIG. 21b shows a perspective view of an embodiment of the laser pointer housing shown in FIG. 21a; the view showing the front-top right portions.

FIG. 22 shows an embodiment of a threaded fastener having a grip handle.

FIG. 23a shows a partial view of an embodiment of a first clamp flange.

FIG. 23b shows an embodiment of a first clamp flange.

FIG. 23c shows an embodiment of a second clamp flange.

FIG. 24 shows an embodiment of hooked mounting adaptor from a perspective-back view.

FIG. 25 shows a fastening strip coupled to a wall and a back view of a target.

FIG. 26 shows a system that includes an interactive target.

FIG. 27 shows a system that includes an interactive target; [system showing an interactive result of the target through a coded scoring algorithm developed through an app accompanying the operation of the apparatus.]

FIG. 28 shows a system that includes a forefoot pressure monitor, a heel pressure monitor, a chest angle sensor, a chin angle sensor, and a head crown angle sensor.

In order to describe the manner in which the above-recited and other advantages and features can be obtained, a more particular description will be rendered by reference to specific implementations thereof which are illustrated in the appended drawings. Understanding that these drawings depict only some implementations and are not therefore to be considered to be limiting of its scope, implementations will be described and explained with additional specificity and detail through the use of the accompanying drawings in which.

### DETAILED DESCRIPTION OF THE INVENTION

Examples extend to apparatus, methods, and systems for prompting a user to alter their posture.

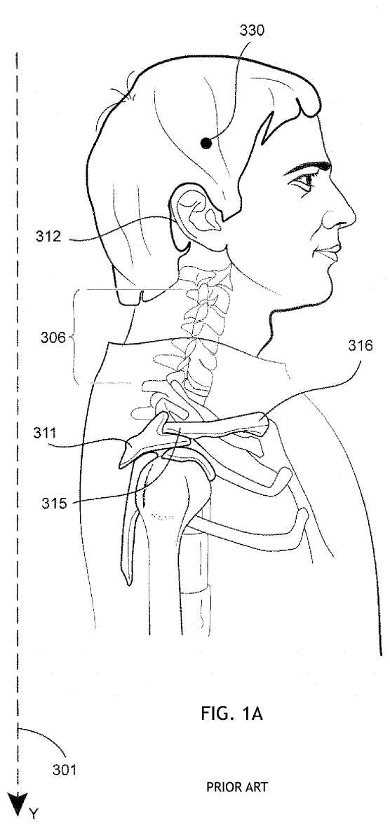

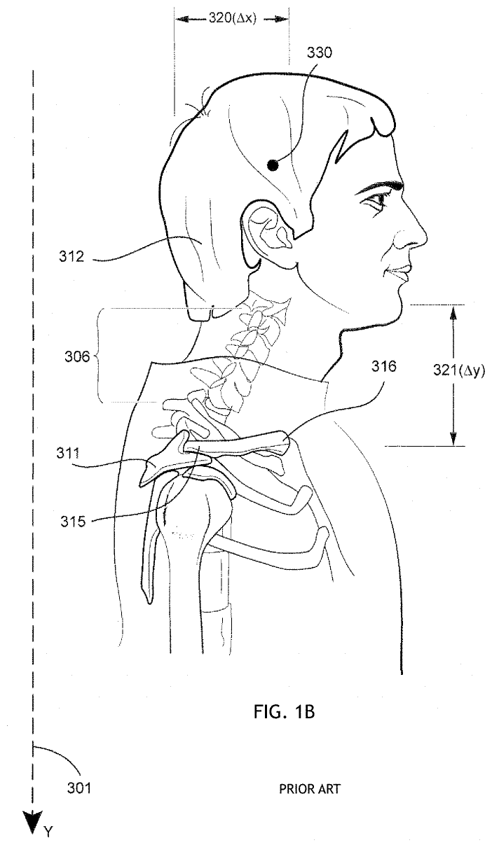

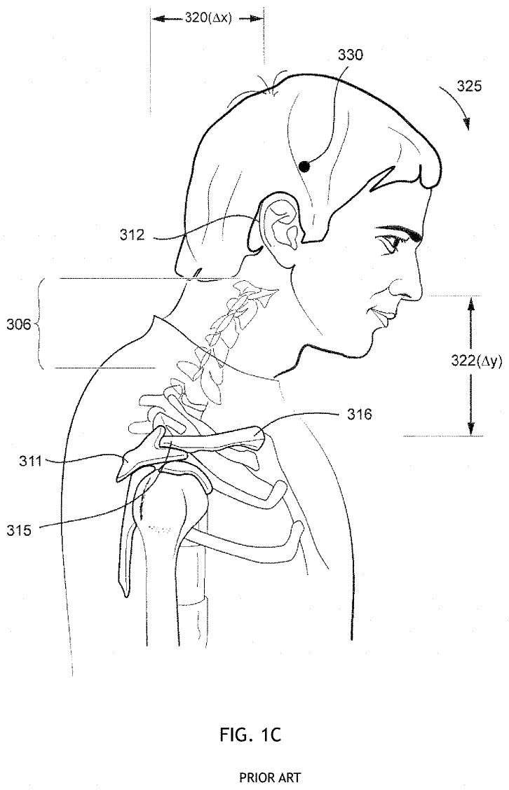

FIGS. 1(a)-1(c) illustrate good and poor posture with respect to the head and neck relationship. As can be seen in FIG. 1(a), a head in the correct posture position is situated above the shoulders (311), with minimal strain to the neck muscles and associated connective tissues. If a person is standing properly, a person's shoulders (311), ears (312), hips, knees, and ankles should be stacked above each other, with an imaginary straight line being able to be drawn from the top of the head, through the body's center, and down to the bottom of the feet. Correct head position related to posture can be determined by comparing the lower ear lobe (312) with the acromioclavicular joint (315) (where the collarbone attaches to the shoulder). These two body landmarks should be lined up in a substantially vertical plane (301). See U.S. Pat. No. 9,406,211.

Contrast FIG. 1(a) with FIG. 1(b), where the head is in a forward position denoted as being forward a distance (ΔX) 320 relative to FIG. 1(a), and the ear (312) and collarbone (316) may not be aligned vertically. In the orientation illustrated in FIG. 1(b), the neck muscles are strained, the connective tissues are stretched, and the cartilage and bones are compressed due to the increased force caused by the forward head position, denoted as a distance 321 (ΔY). For instance, based on an average head, the head weighs approximately 12 lbs. in the correct upright position. As the head moves forward, the weight (downward force) of the head increases: two inches of forward head movement increases the weight of the head to approximately 32 lbs. and three inches of forward head movement increases the weight of the head to approximately 42 lbs. of force. The said increased weight may be supported by the muscles of the neck and upper back. Additionally, the forward movement of the head shifts its center of gravity 330 forward, causing the upper body to drift backward, the hips to tilt forward, and strain to the middle and lower back as a result. Further, the additional force of forward head positions may cause the bones of the neck to compress the associated cartilages, blood supplies, and nerves. See U.S. Pat. No. 9,406,211.

FIG. 1(c) illustrates a forward head posture whereby a person has also tilted their head downwards to look at a handheld electronic device. Postural issues associate with forward head position issues may be compounded by the user additionally looking down at the device, causing angular head tilt 325 in addition to the forward lateral movement 320 associated generally with forward head resulting from poor posture. Thus, the forward lateral movement 320 in conjunction with the head tilt 325 further moves the head's center of gravity 330. The tilt 325 further compresses the bones, muscles, etc. (denoted as a distance (ΔY) 322), and compounds issues with pinched nerves and cartilage discs. Prolonged use of handheld devices while exhibiting this improper posture may result in permanent spinal and muscle deformities. See U.S. Pat. No. 9,406,211.

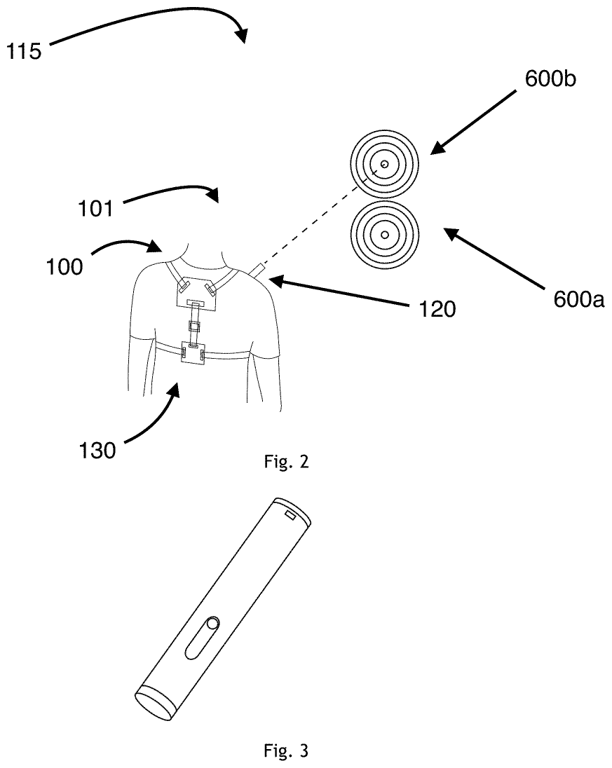

Referring to the schematic of FIG. 2, a biofeedback system (115) for altering posture refers to an illuminable orthostatic reporting apparatus (101) for altering posture that may be worn by a user; the illuminable orthostatic reporting apparatus (101) including a laser pointer (120) coupled to an orthostatic reporting assembly (100); some components of the apparatus shown: the chest mount harness assembly (130), an adjustable housing assembly (400) that is not shown since it is obscured by the back of the user and includes laser housing hardware with adjustment mechanisms, and a laser pointer (120). The biofeedback system (115) includes a first target (600a) and a second target (600b).

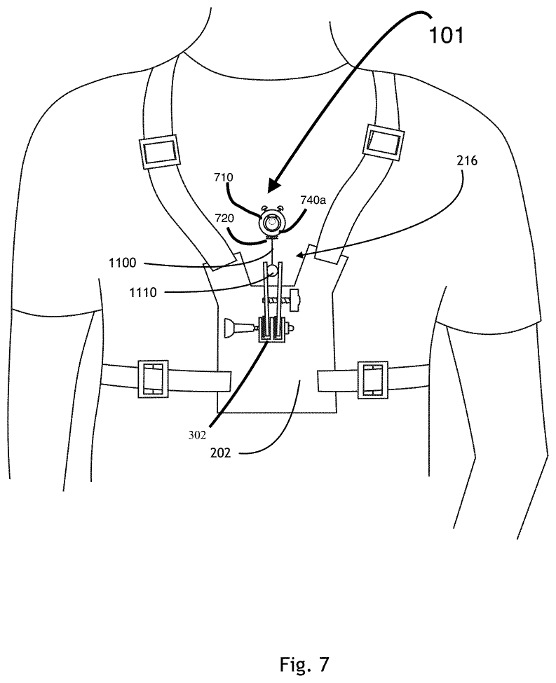

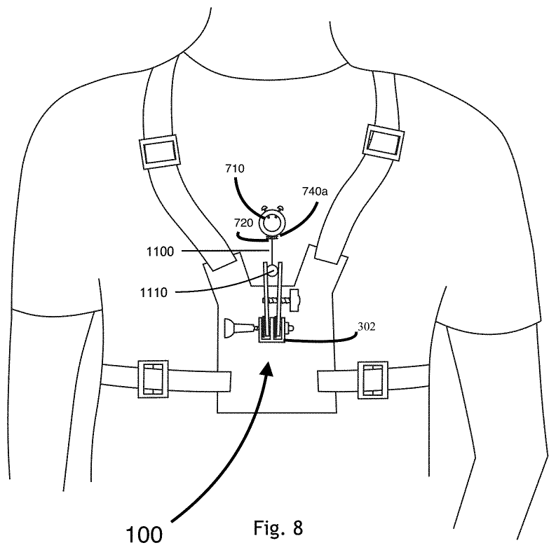

The disclosure generally relates to an orthostatic reporting assembly (100), see FIG. 8 for example, for prompting a user of the apparatus to alter the user's posture. In some embodiments, specifically, the disclosure relates to an illuminable orthostatic reporting apparatus (101), see FIG. 7, which may include an orthostatic reporting assembly (100), the orthostatic reporting assembly (100) may be coupled to a laser pointer (120) disposed within a portion of an adjustable housing assembly (400), and the orthostatic reporting assembly (100) may be releasably coupled to a chest mount harness assembly (130) at a height so as to hold a laser pointer at the level of the sternum, nearby the sternum, above the sternum, or below the sternum. The orthostatic reporting assembly (100) may be configured to be strapped to a person's chest and may provide a mountable surface for an adjustable laser housing assembly 216 upon which a laser pointer or illuminating device is mounted. The orthostatic reporting assembly (100) may provide a substantially stable platform that may minimize swaying of the laser, and thereby facilitate the provision of visual feedback to a user; the visual feedback may be useful for a user training to strengthen the user's postural musculature. A strengthened postural musculature may contribute to a healthy posture. In some embodiments, the chest mount harness assembly (130) may be adapted so as to be placed at a body location other than the sternum such as: an arm, a torso, a forehead, etc. A biofeedback system (115) for using the illuminable orthostatic reporting apparatus (101) is disclosed. Nonlimiting embodiments of the disclosed biofeedback system (115) may include: a) illuminable orthostatic reporting apparatus (101,100, and 130) disposed upon a user and a number of targets (600) mounted on a fixed surface, b) an illuminable apparatus assembly including a laser pointer (120), c) a system that includes an illuminable apparatus assembly and a target, and d) apparatus, laser, and target to prompt a user to alter posture.

The present invention may include a postural strengthening apparatus for providing health benefits.

Referring to FIG. 3, an embodiment of a laser pointer (120) is depicted.

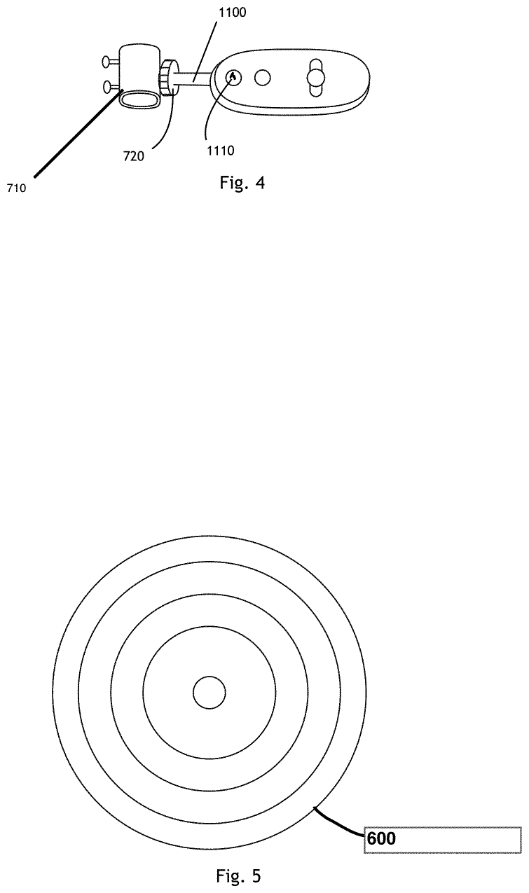

Referring to FIG. 4, an embodiment of a laser pointer housing (710) coupled to a nut (720), which is coupled to a fastening component (403) an adjustable housing assembly (400) mounting hardware that may function as laser mounting hardware.

Referring to FIG. 5, a front view of the target that is mounted on a fixed surface or a specified desired height.

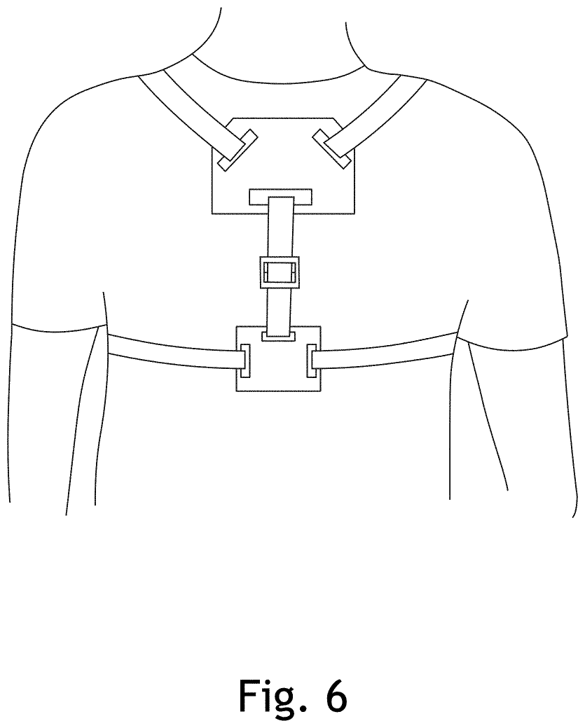

Referring to FIG. 6, a back view of the chest mount harness assembly 130 is depicted.

Referring to FIG. 7, a front view of the illuminable orthostatic reporting apparatus (101) for altering posture is depicted.

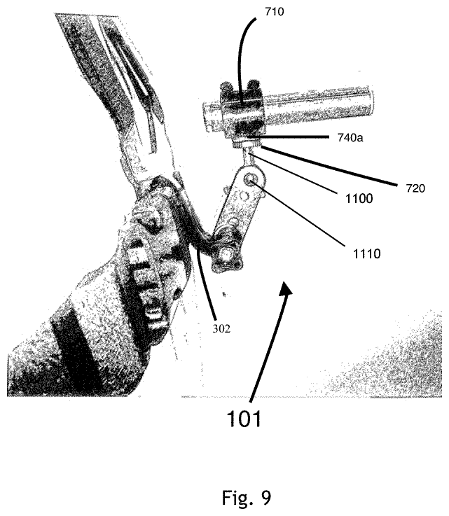

Referring to FIG. 9 a right sided photograph of the illuminable orthostatic reporting apparatus assembly.

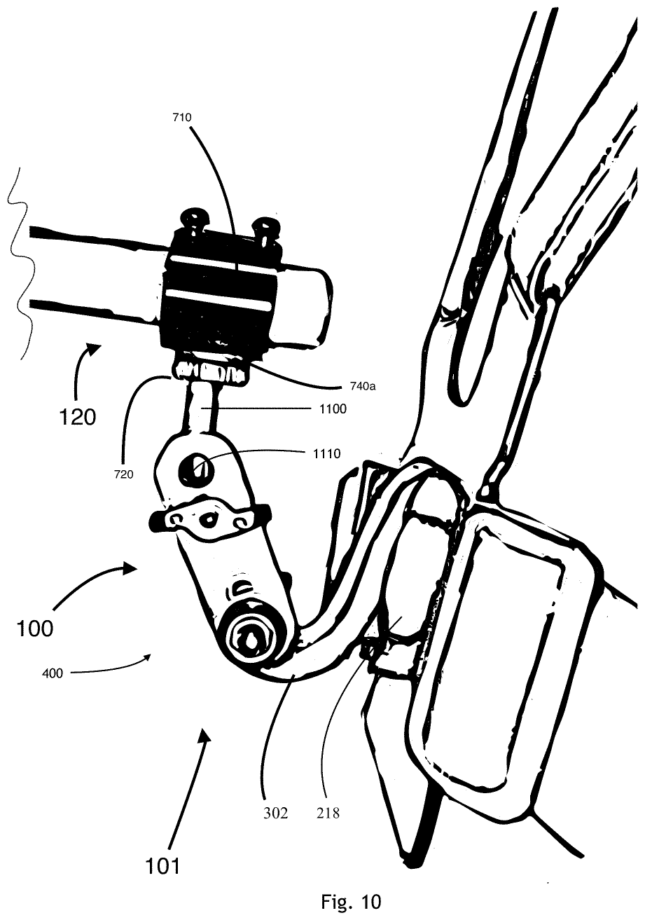

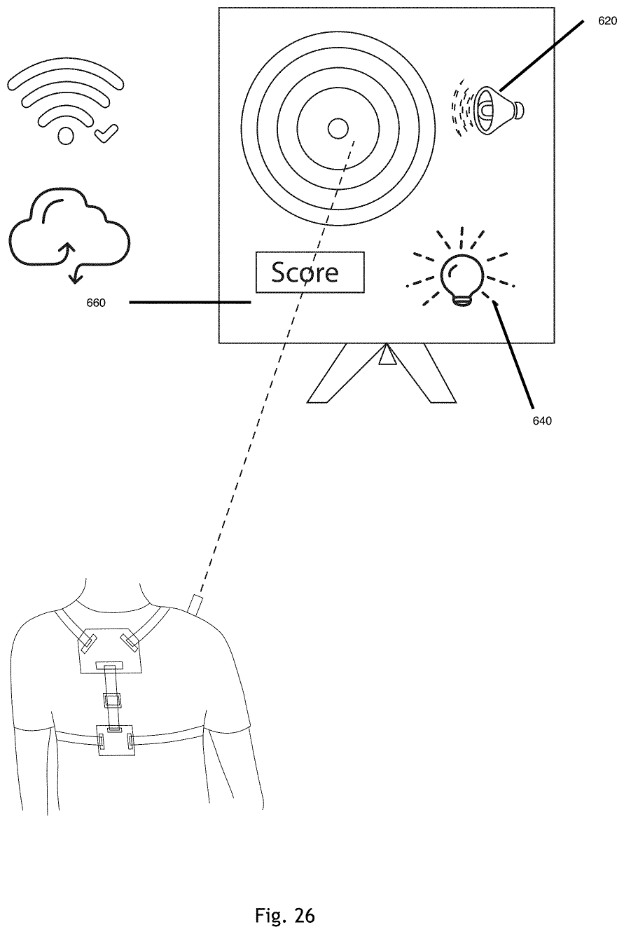

Referring to FIG. 10, an illuminable orthostatic reporting apparatus (101) left side angle view for orthostatic training. FIG. 26 depicts an embodiment of a target (600).

Referring to FIG. 2, a perspective side view of an embodiment of a orthostatic reporting apparatus (110) for altering posture with three components of the apparatus are shown: the chest mount harness assembly (100), an adjustable housing assembly (400) (400) that may include mounting hardware with adjustment mechanisms for adjusting the position of a laser that has been mounted in the adjustable housing assembly (400) (400) or for tightening the grip of a housing, of the adjustable housing assembly (400), on a laser that has been disposed within the housing. The adjustable housing assembly (400) (400) may include balls and swivels, and the laser.

FIG. 3: A top view of the orthostatic reporting apparatus for altering posture with three components of the apparatus is shown, including embodiments of the harness, the adjustable housing assembly (400) and the laser.

FIG. 4: A depiction of some components of the orthostatic reporting apparatus for altering posture with three components being viewed separately. The harness, the mounting laser hardware with adjustment mechanisms and the laser are depicted.

FIG. 5: The schematic depicts a user that is maintaining the laser pointer in a position so as to be aligned with the target during movement such as walking. This is one example of the postural strengthening functions of the apparatus.

FIG. 6: A photograph of an embodiment of an adjustable housing assembly (400) (400) is shown.

FIG. 7: A schematic of component of the adjustable housing assembly (400) that is shown in FIG. 6; an embodiment of a harness (130b) is shown; the harness 130b being able to be strapped around a user. Main plate (202) may be configured to detachably attach or be inseparably attached to an adjustable laser housing assembly (216), which may be an assembly of a plurality of adjustable components.

Referring to FIG. 10, the illustration depicting the appendage mechanism (302) to the chest mount harness assembly holding the illuminable (120) laser housing (710) maneuvered by a nut (720) (740a) and a fastenable (1100)-swivel locking ball-joint (1110) tightly held in a protruded position by a swivel locking ball-joint clamp (100) (400) mainly fastened on to the main plate of the orthostatic reporting apparatus.

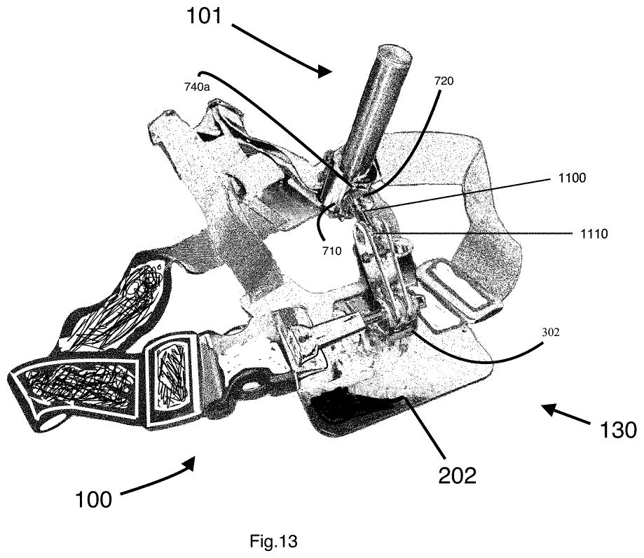

FIG. 13 depicts a birds-eye view of an embodiment of an illuminable orthostatic reporting apparatus (101) for altering posture comprising: of a laser housing (710) attached by a nut (720) and a fastenable swivel locking ball joint (1110) enclosed by a swivel locking ball-joint clamp (1110) fixed to the main plate (202) by a chest mount harness assembly (302).

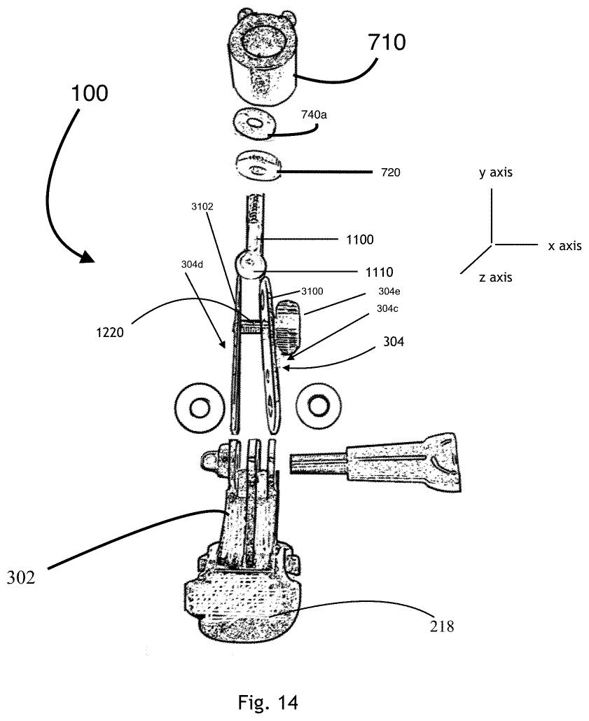

Referring to FIG. 14, depicts the protruded mechanism carrying the illuminable orthostatic reporting apparatus (710) maneuvered by a fastener (1100) swivel locking ball-joint (1110) joined together by a nut (720) and washer (740a) the use of a laser housing connected by an appendage mechanism (302), connected by a swivel locking ball-joint clamp (304) tightly locked by a fastener joint connector bolt (1220).

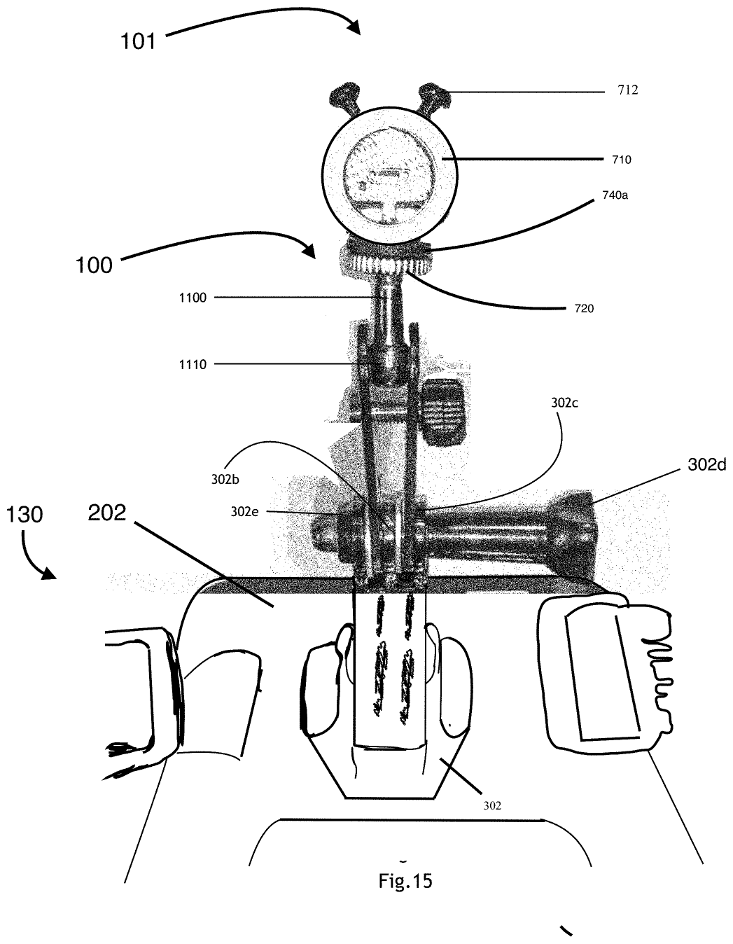

Referring to FIG. 15, a chest mount harness assembly (130) is shown; the chest mount harness assembly (130) may include a main plate (202) and a fastening connector (218) of the main plate (202), the fastener (206) may have at least one nut (208), a bolt (210) may be insertable into at least one nut (208), a flange assembly (212) may be inseparably coupled to the bolt (210) or at least one nut (208), a ball element (214) may be couplable by the flange assembly (212); the ball element (214) may be couplable by the flange assembly (212); an adjustable laser housing assembly (216) may be couplable to the ball element (214).

FIG. 18, shows the bottom view of the appendage mechanism (302) having three encarved curves (302c) allowing the appendage to be adjusted and maneuvered as desired by the user.

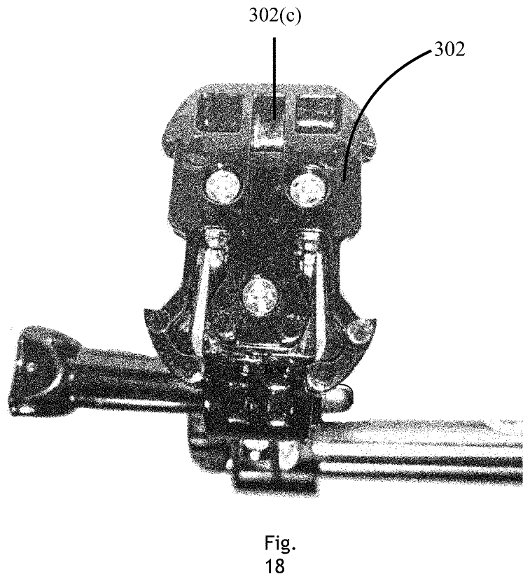

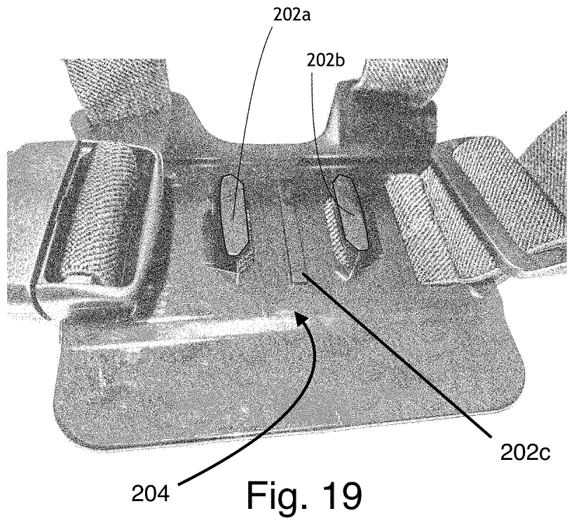

FIG. 19 depicts a housing 204 coupled to a portion of a chest mount harness assembly 130; the fastening connector (218) may include a first flange (202a), a second flange (202b), and a channel flange (202c), the fastening connector (218) may be configured to detachably couple with clasps of a quick-release fastener; the first flange 202a and the second flange 202b may be substantially L-shaped, each having an overhanging flange for engaging with a portion of a quick-release clasp.

FIG. 9 depicts a closeup of the embodiment shown in FIG. 8.

FIG. 10 depicts side view of the embodiment show in FIG. 8.

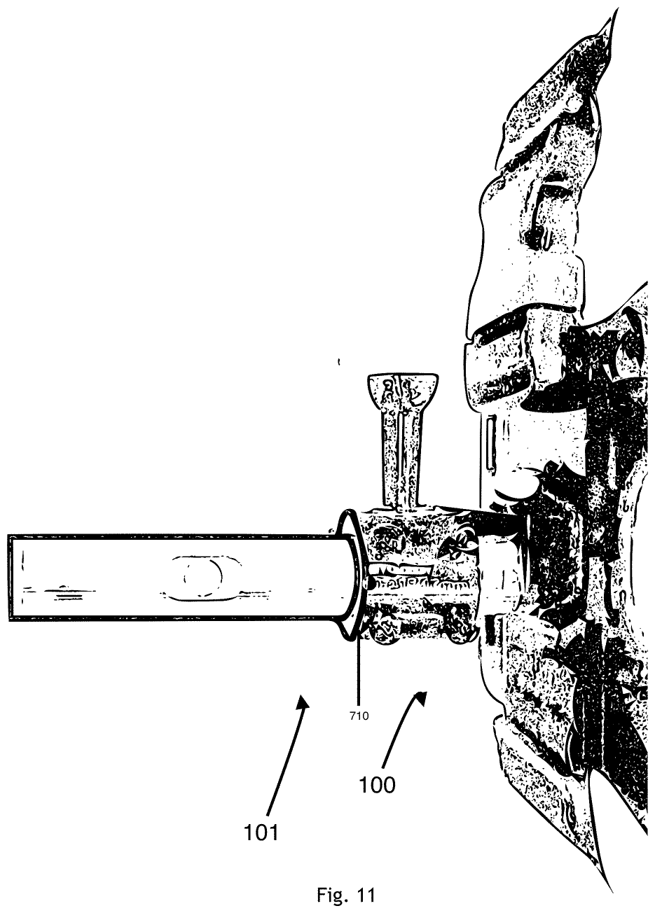

FIG. 11 depicts an embodiment of a laser coupled to a charger.

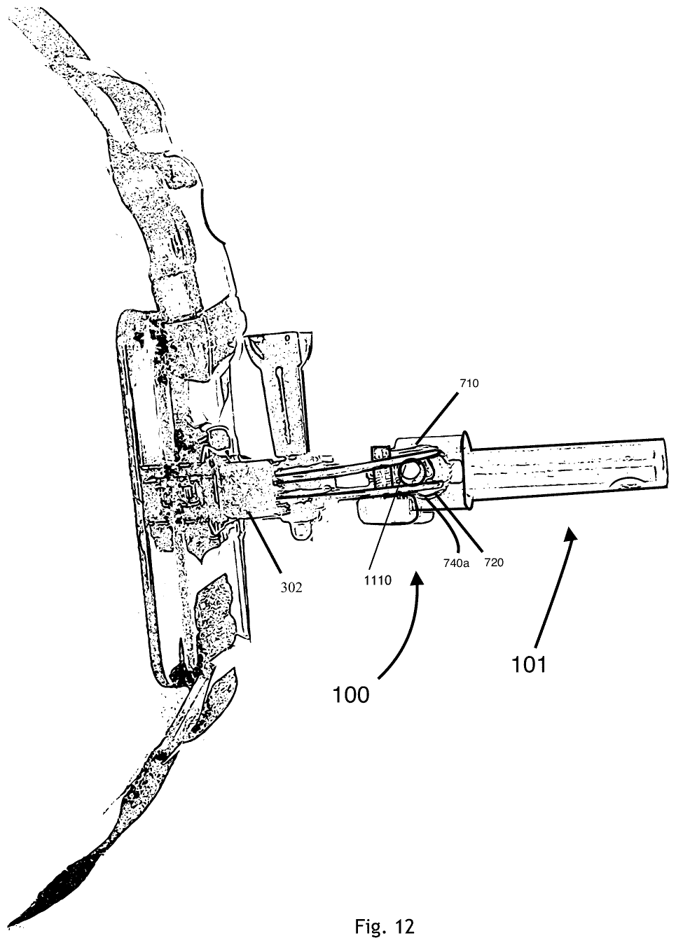

FIG. 12 depicts another view of the laser of FIG. 11.

FIG. 13 depicts a harness coupled to a plate.

FIG. 14 depicts components that are show in FIG. 6.

FIG. 15 depicts components that are show in FIG. 14.

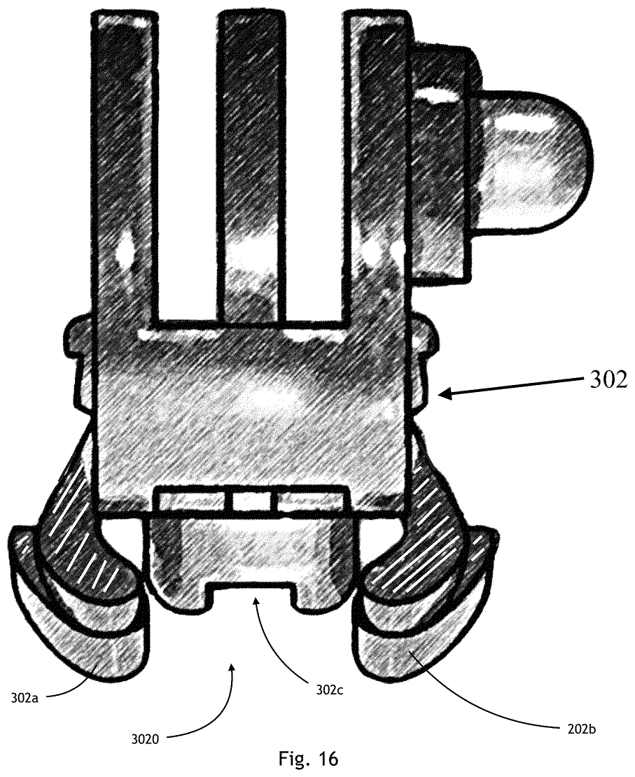

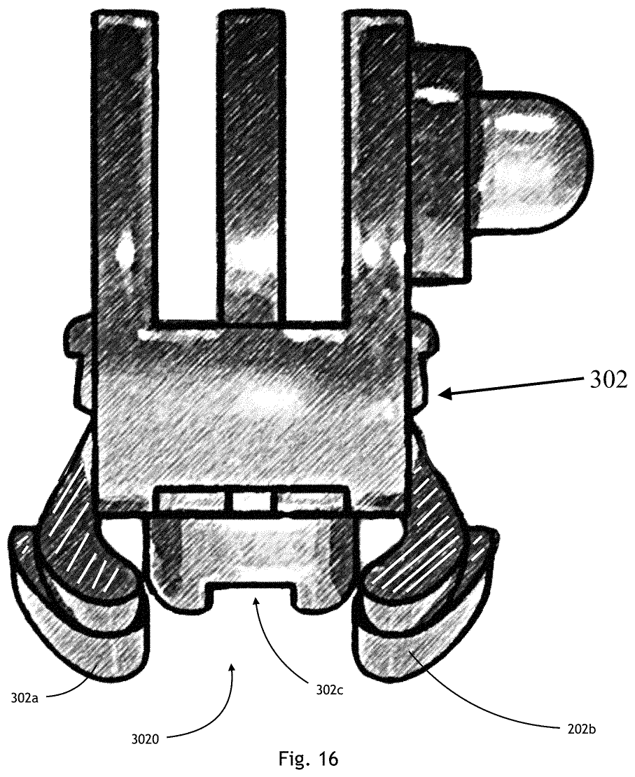

FIG. 16 depicts components that are show in FIG. 14.

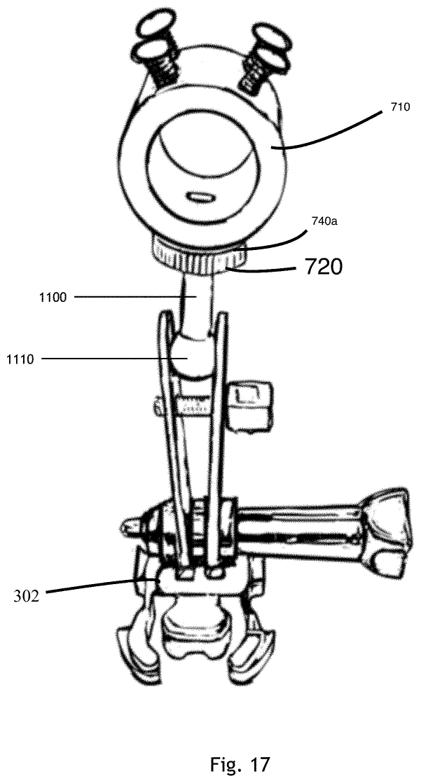

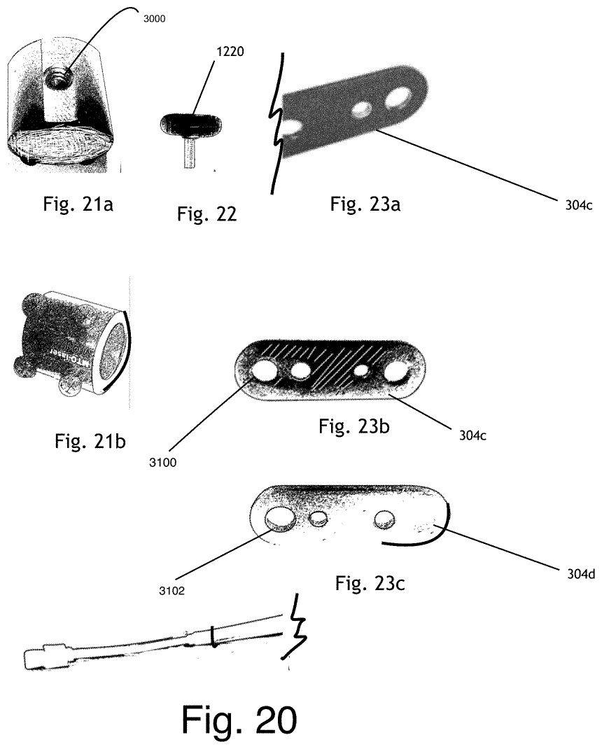

FIG. 17 to FIG. 22 shows a user using the apparatus.

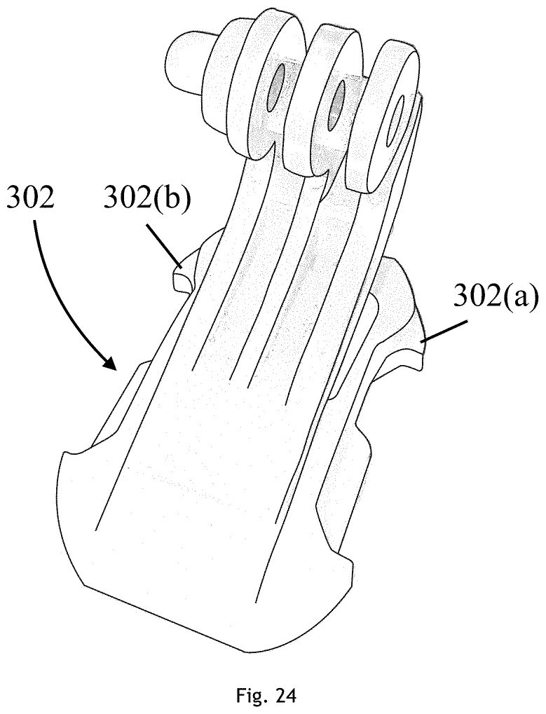

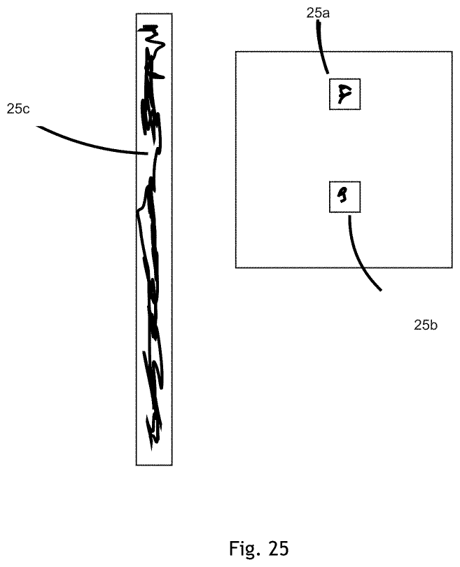

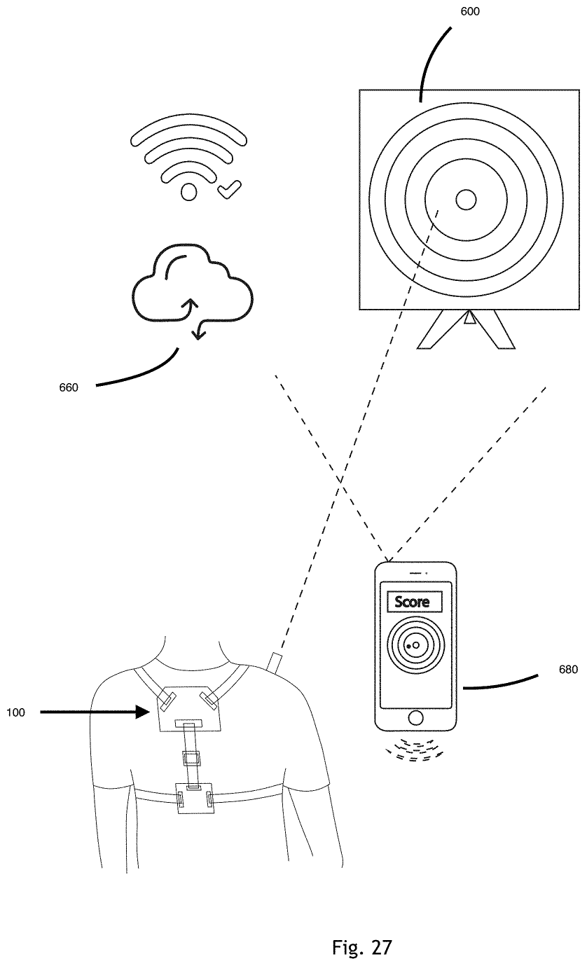

FIGS. 23 to 27 shows various perspective-views of what has already been shown, including the back side of an embodiment of a main plate (202).

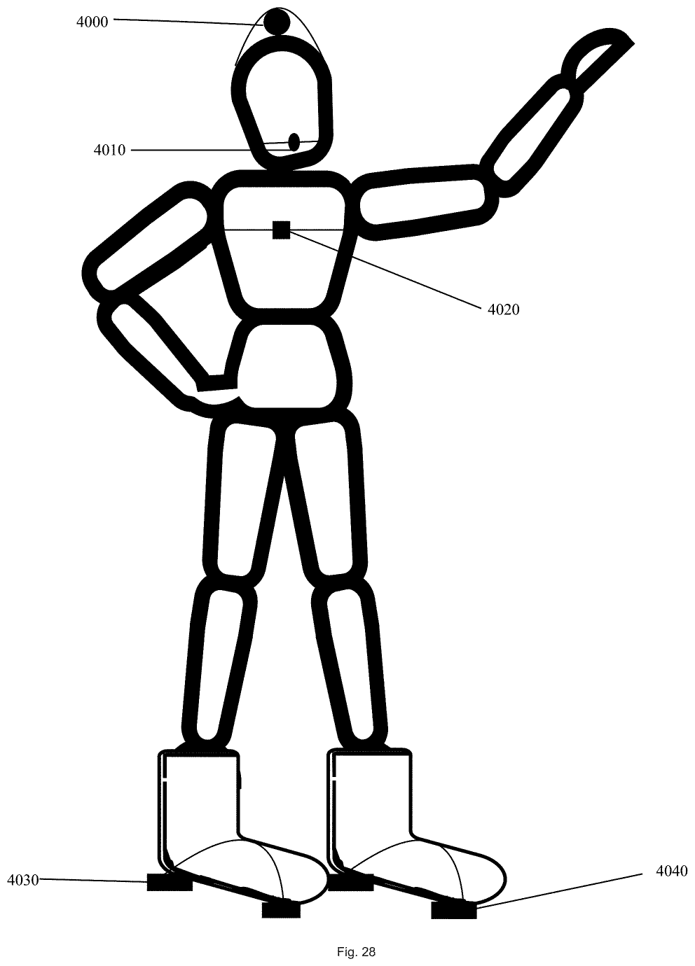

FIG. 28 depicts the general location of components that make up part of the orthostatic reporting system: a heel pressure monitor; a forefoot pressure monitor; a chest angle sensor; a chin angle sensor; and a head crown angle sensor.

In some examples, the target may have sensors for receiving the laser light; the sensors on the target may sense a laser beam and using a chip record the number of times or length of period that the laser beam has been pointed at a specific area of the target; and the chip may record stats and send them to a device such as a server or a smart phone; alternatively, a paper target or other target may be used that does not have a sensor, and a smart phone device may be pointed at the target and the smart phone may have an non-transitory storage medium that may store computer code instructions for analyzing input from a camera of a smart phone to determine the accuracy of a user as measured by the amount or amount of time that laser pointer lights are directed at a specific area of the target. In some embodiments when the laser light may be detected at a position that is suboptimal then the apparatus assembly may produce a sound that may signal the user a need to correct their posture. Alternatively, in some examples, the target may have a sensor that may be located in the target area such that when the user walks in such a way as to cause the laser light that is emitted by the laser to contact the target area, then the sensor may signal the apparatus or a portion of the apparatus to emit a feedback sound or series of feedback sounds, thereby providing feedback and optionally keeping score of the number of times that the user successfully completed the task by walking with a certain posture that allowed the laser to point the laser light in an aligned arrangement, such as striking the bullseye of the target.

In more detail, still referring to the invention of the figures, the harness may have straps with adjusting mechanisms so that the harness may be altered or sized so as to be worn or comfortably worn by different-sized people at different times. The base may be a portion of the orthostatic reporting apparatus for altering posture, and it may be a secure, mountable base that may receive the adjustment mechanisms. Furthermore, the adjustment mechanisms may be used when a user is using the orthostatic reporting apparatus for altering posture to adjust the laser to the target. When a user has started wearing the apparatus, the adjustment mechanisms may be adjusted by the user so as to move the laser diagonally, vertically, or horizontally so that a user's posture may be properly adapted for the target and the target's location. Generally, an adjustment mechanism may be used by a user or a trainer who is assisting the user by placing the apparatus on the user so that people of different height and sizes may alter the configuration of the apparatus to fit their height or size. The mounting laser hardware may have a set of fasteners to keep the laser secure. The laser may be the training component that strikes the target for the feedback needed for a successful laser posture session.

In one example, a harness may be strapped around the person or user to stabilize the adjustable housing assembly (400), which may be plastic, to the person's chest. The adjustable housing assembly (400) may be configured to receive the mounting components of the Orthostatic reporting apparatus for altering posture, also referred to as parts of the apparatus. The adjustment mechanism is shown mounted to a base, which may be a plastic base. The adjustment mechanism may be attachable or connectable to the base. Now, the mounting laser hardware, which may be a housing configured to receive the laser, is shown. Finally, the laser that may be secured by the adjustable housing assembly (400) is shown when it is in a secured position whereby it is secured to mounting laser hardware. Lastly, target is depicted when it is secured to a wall, that may be stationary.

In another example, the harness may have straps with adjusting mechanisms so that the harness may be altered or sized so as to be worn or comfortably worn by different-sized people at different times. The adjustable housing assembly (400) may be a portion of the orthostatic reporting apparatus for altering posture, and may be a secure, mountable base that may receive the adjustment mechanisms. Furthermore, the adjustment mechanisms may be used when a user is using the orthostatic reporting apparatus for altering posture to adjust the laser to the target. When a user has started wearing the apparatus, the adjustment mechanisms may be adjusted by the user so as to move the laser diagonally, vertically, or horizontally so that a user's posture may be properly adapted for the target and the target's location. Generally, an adjustment mechanism may be used by a user or a trainer who is assisting the user by placing the apparatus on the user so that people of different height and sizes may alter the configuration of the apparatus to fit their height or size. The mounting laser hardware (400) may have a set of fasteners to keep the laser secure. The laser may be the training component that strikes the target (600) for the feedback needed for a successful laser posture session.

The construction details of the invention as shown in the figures: the harness may be made of an elastic strapping material that may have the ability to hold the adjustable housing assembly (400) in place for proper functioning. The various components of the orthostatic reporting apparatus for altering posture can be made of different materials.

The construction may include a harness that may be made of an elastic strapping material that may have the ability to hold the orthostatic reporting apparatus for altering posture in place for proper functioning. The various components of the orthostatic reporting apparatus for altering posture can be made of different materials.

A method may include using one or more targets; a method may include using a first target with a first diameter followed by using a second target with a second diameter; wherein the second diameter may be less than the first diameter. A method may include using a first target that is placed between 3.5 feet and 4.5 feet above the ground, which may be followed by placing a second target between 4.5 feet and 5.5 feet above the ground; in some preferred methods; the target is first placed substantially 4 feet above the ground; a user may then walk towards the target while wearing the apparatus; then the user may then walk towards a target that is substantially 5 feet above the ground or above the height at which the first target was placed.

The apparatus may also be an assembly that may include two or more lasers, a second laser may be placed substantially vertically aligned or at an angle. In some methods, a track comprises a lane of two substantially parallel lines that are aligned along the ground. In some methods, a user may be wearing a harness that may have two or more lasers, and the user may walk towards two or more targets with the purpose of aligning the first laser with the first target and aligning the second laser with the second target. In some methods, a user may wear protective glasses and may wear the apparatus while walking towards a mirror.

The present invention may include a posture strengthening apparatus for many health benefits.

Orthostatic Reporting Apparatus for Altering Posture

Posture improves your bone health, it improves your muscle health, it improves your blood circulation health, it improves your lung respiratory health just to name a few. This apparatus gives visual feedback to help improve posture and strengthen postural muscles all the while realigning the spine and pelvic bones. [“This apparatus gives visual feedback to make the user aware of current maladaptive posture. The visual feedback by the laser light pointing to the fixed target allows posture improvement awareness thus making the user recognize the need to consciously correct the user's spine whilst the apparatus is worn”.] Use of the apparatus for altering posture may, even operate in brief periods of time, may result to lasting effects on a user's posture.

The apparatus may include a number of portions, including a securing system, such as a harness, the laser housing hardware with adjustment mechanisms, an adjustable housing assembly (400), a laser, and a target.

The securing system secures the apparatus to a user. The securing system may be a harness used to strap around the person's torso to stabilize the adjustable housing assembly (400). The adjustable housing assembly (400) is used to receive the laser housing hardware with an adjustment mechanism. The adjustment mechanisms may be a part of the adjustable housing assembly (400) to the base. The laser housing hardware receive the laser.

This apparatus is for training the posture which utilizes target and the orthostatic reporting apparatus for altering posture for a successful laser posture session.

The laser could emit a line instead of a point. Multiple lasers could be used requiring additional mounting laser hardware with adjustment mechanisms.

The mounting laser hardware may be a laser housing with a set of fasteners. The adjustment mechanisms may be a ball swivel with easy grip fasteners comparable to wing nuts.

In some embodiments, the laser may be oriented in a different direction so as to form vertical lines of light; or in some embodiments, a plurality of laser housings and a plurality of lasers are included in the apparatus. In some embodiments, the addition of one or more horizontal lines of light generated by one or more additional mounted lasers may provide additional biofeedback and may aid in helping the user keep the apparatus stable during a laser posture session. In some embodiments, the addition of one or more vertical lines of light generated by one or more additional mounted lasers may provide additional biofeedback and may aid in helping the user keep the apparatus stable during a laser posture session. In some embodiments, additional laser housing mounted with one or more lasers may be oriented in a direction other than vertical or horizontal.

Lasers with vertical and horizontal lines create higher stability and thus more helpful feedback to create a more successful laser posture session.

An objective is to keep the laser on the target, more specifically, on a point on the target. This may be done during sitting, standing, and walking. More specifically to be done while in perfect posture or as good as the patient can achieve (weight on heels, sternum up, chin tucked in and being as tall as possible thus engaging the core musculature for spinal traction). All steps can be scribed into one great whole of improving patients' posture.

The securing system may use elastic strapping to adhere to a user's torso. A plastic base may provide a lightweight system to help the laser posture session to be more successful and not too distracting with a heavy substance such as metal. The adjustable housing assembly (400) may include mounting laser hardware made of steel or plastic to keep it durable and easy to use. The adjustable housing assembly (400) may be a round hollow ring used to house the laser. Other materials may be used to make the apparatus lighter and cheaper to manufacture.

An apparatus for altering posture is described. The apparatus incudes a securing system, an adjustable housing assembly (400), a laser, and a target. The securing system secures the apparatus to a user. The aiming system is physically connected to the securing system such that the adjustable housing assembly (400) is securable to a sternum. The laser is mechanically connected to the aim system and projects a point of light. The target is used to provide a point where the point of light projected by the laser is to be projected.

The securing system may include any method of securing the apparatus to a user. In one example, the securing system is a series of straps that wrap around the person. The straps may be made out of elastic or a hook and fastener system. In another example the securing system may be made of hooks that secure the apparatus to a user.

The adjustable housing assembly (400) may be physically connected to the securing system. The adjustable housing assembly (400) may include a mountable base and an adjustable mechanism. The mountable base may physically connect the adjustable mechanism to the securing system. The mountable base may include an adjustable mechanism that allows a laser to be adjusted with greater precision than adjustments made to the securing system.

A laser, mechanically connected to the adjustable housing assembly (400), is physically using the adjustable housing assembly (400). The aiming system allows the laser to be aimed at a particular point or target.

The target may be a point, a distance away from the laser, where the laser may be focused.

A method for improving posture is described.

An apparatus for altering posture may be secured to a person. That securing may involve wrapping a number of straps around the person and securing them using elastic, hook-and-fastener fabric, hooks, D-rings or the like.

The person may be placed a distance from the target of the apparatus for altering posture. This may allow the person to focus on the target while holding the laser in place using a desired posture.

The apparatus for altering posture is adjusted using the adjustable housing assembly (400) such that the laser is projected on the target of the apparatus for altering posture when the person has the desired posture.

In one example, the person may walk, while maintaining the point of light at a consistent place on the target of the apparatus for altering posture. In another example the person may sit in place while maintaining the light from the laser on the target.

In more detail, still referring to the invention of Figure five, the harness (130b) may have straps with adjusting mechanisms so that the harness may be altered or sized so as to be worn or comfortably worn by different-sized people at different times. Base (200) may be a portion of the orthostatic reporting apparatus for altering posture, and it may be a secure, mountable base that may receive the adjustment mechanisms (300). Furthermore, the adjustment mechanisms may be used when a user is using the orthostatic reporting apparatus for altering posture to adjust the laser to the target (600). When a user has started wearing the apparatus, the adjustment mechanisms may be adjusted by the user so as to move the laser diagonally, vertically, or horizontally so that a user's posture may be properly adapted for the target and the target's location. Generally, an adjustment mechanism may be used by a user or a trainer who is assisting the user by placing the apparatus on the user so that people of different height and sizes may alter the configuration of the apparatus to fit their height or size. The mounting laser hardware (400) may have a set of fasteners to keep the laser (500) secure. The laser (500) may be the training component that strikes the target (600) for the feedback needed for a successful laser posture session.

Referring to the embodiment shown in FIG. 1: the harness (130b) may be made of an elastic strapping material that may have the ability to hold the orthostatic reporting apparatus for altering posture in place for proper functioning. The various components of the orthostatic reporting apparatus for altering posture can be made of different materials.

A method may include using one or more targets; a method may include using a first target with a first diameter followed by using a second target with a second diameter; wherein the second diameter may be less than the first diameter. A method may include using a first target that is placed between 3.5 feet and 4.5 feet above the ground, which may be followed by placing the target between 4.5 feet and 5.5 feet above the ground; in some preferred methods; the target is first placed substantially 4 feet above the ground; a user may then walk towards the target while wearing the apparatus; then the user may then walk towards a target that is substantially 5 feet above the ground or above the height at which the first target was placed.

The apparatus may also be an assembly that may include two or more lasers, a second laser may be placed substantially vertically aligned or at an angle. In some methods, a track comprises a lane of two substantially parallel lines that are aligned along the ground. In some methods, a user may be wearing a harness that may have two or more lasers, and the user may walk towards two or more targets with the purpose of aligning the first laser with the first target and aligning the second laser with the second target. In some methods, a user may wear protective glasses and may wear the apparatus while walking towards a mirror. Illuminable means capable of being illuminated.

The present invention may include a posture strengthening apparatus for many health benefits. This apparatus gives visual feedback to help improve posture and strengthen postural muscles all the while realigning the spine and pelvic bones.

Some embodiments may have at least three parts: the harness, the laser housing hardware with adjustment mechanisms, the laser and target.

The harness may be used to strap around the person's torso to stabilize the base. The base may be used to receive the laser housing hardware with adjustment mechanisms. The adjustment mechanisms may attach to the base. The laser housing hardware may receive the laser.

This orthostatic reporting apparatus may be used for training the posture which utilizes a target and the orthostatic reporting apparatus for altering posture during a laser posture session.

The laser may emit a line of light instead of a point. Multiple lasers could be used which may require additional mounting laser hardware with adjustment mechanisms.

The parts could be remanufactured with completely different configurations.

Lasers with vertical and horizontal lines may be used to create higher stability and thus may provide additional feedback to create a more successful laser posture session.

In some methods, the user tries to keep the laser on the target more specifically on the bullseye. This may be done during sitting, standing, and walking. In some methods, the user walks towards the target wearing the apparatus in a perfect posture or as good as the patient can achieve posture (weight on heels, sternum up, chin tucked in and being as tall as possible thus engaging the core musculature for spinal traction).

Dancers and other performers and other activities may use the device to improve their posture.

The harness may include elastic strapping to adhere to one's torso. The base may be plastic and may be lightweight. In some embodiments, a base that is substantially lightweight, when compared to a metal base, may be useful. The mounting laser hardware may be made of steel and plastic. The mounting laser hardware may be durable and easy to use. The laser housing hardware may be a round hollow ring used to house the laser. This could be made of other materials lighter and cheaper to manufacture.

In some methods, a user may set the user's posture in good form by putting weight on heels, keeping the sternum up, tucking in the user's chin and being as tall as possible thus engaging the core musculature for spinal traction.

This apparatus may be used during a 10-15-minute strengthening session using laser guided feedback for a successful laser posture session. This may be used to improves strength to postural muscles and improves body awareness for lasting corrective postural gains.

The present described aspects may be implemented in other specific forms without departing from its spirit or essential characteristics. The described aspects may be considered in all respects only as illustrative and not restrictive. The scope is, therefore, indicated by the appended claims rather than by the foregoing description. All changes which come within the meaning and range of equivalency of the claims are to be embraced within their scope. Various alternative configurations of the components that are disclosed herein is contemplated.

What is claimed is:

1. An illuminable orthostatic reporting apparatus comprising: an orthostatic reporting assembly comprising an adjustable housing assembly, and a chest mount harness assembly coupled to the adjustable housing assembly; and a pivotable laser pointer disposed within a laser pointer housing of the adjustable housing assembly, wherein the adjustable housing assembly is rotatable around a horizontal axis and rotatable along a vertical axis and z axis, the pivotable laser pointer coupled to interact with the assembly.

2. The illuminable orthostatic reporting apparatus as of claim 1, wherein the adjustable housing assembly is rotatable around vertical axis and z axis by at least 10 degrees.

3. The illuminable orthostatic reporting apparatus as of claim 1, wherein the adjustable housing assembly of the illuminable orthostatic reporting apparatus further comprises a pivotable pointer housing assembly comprising a laser pointer housing.

4. The illuminable orthostatic reporting apparatus as of claim 1, wherein the pivotable pointer housing of the pivotable pointer housing assembly of the adjustable housing assembly further comprises a threaded hole coupled to a fastener and an articulating clamp assembly, the articulating clamp assembly further comprising: a plurality of clamp flanges configured to couple together by a clamp assembly fastener.

5. The illuminable orthostatic reporting apparatus as of claim 1, wherein the fastener coupled to the threaded hole and coupled to the articulating clamp assembly is a ball head fastener comprising a ball head portion and a threaded bolt portion, wherein the threaded bolt portion is mateably coupled to the threaded hole of the laser pointer housing and the ball head portion is rotateably disposed between a third aperture of the first clamp flange and a fourth aperture of the second clamp flange; wherein the chest mount harness assembly comprises a main plate, the main plate comprising a fastening connector of the main plate; wherein a hooked mounting adaptor releasably coupled to the fastening connector and to the articulating clamp assembly; wherein the hooked mount adaptor is configured to receive the first clamp.

6. The illuminable orthostatic reporting apparatus of claim 1 wherein the pivotable laser pointer housing assembly comprises a laser pointer fastened to the laser pointer housing further comprising at least one fastener that holds the laser pointer in place inside the laser pointer housing.

7. The illuminable orthostatic reporting apparatus of claim 1 wherein the pivotable laser pointer housing assembly comprises a laser pointing housing further comprising a fastener ball joint fastened to the laser pointer housing by a nut and a washer.

8. The illuminable orthostatic reporting apparatus of claim 1 wherein articulating clamp assembly comprises a clamp assembly fastener and a fastener joint connector bolt that helps keeps level and stable the pivotable laser pointer housing assembly.

9. The illuminable orthostatic reporting apparatus of claim 1 wherein the hooked mounting assembly comprises a knob covered fastener and knob covered fastener nut that fastens the hooked mounting assembly to the articulating clamp assembly.

10. An illuminable orthostatic reporting system comprising: a first target, the first target having a bullseye; and a second target having a bullseye, the second target being positioned above the first target.

11. The illuminable orthostatic reporting system of claim 9, wherein the first target comprises a light sensor positioned at a center of the bullseye of the first target, the light sensor facing the user, the light sensor being operably coupled to a system comprising: at least one of a sound emitting device, a light-emitting device, and a display device; a processor; and a non-transitory memory storage storing instructions that when executed by the processor cause the processor to perform operations, the operations comprising: a receiving during a first time period, via the light sensor, light intensity data corresponding to an amount of light reflected from the bullseye of the second target; a determination of a length value of a subset time period of the first time period for which the light intensity data corresponding to the amount of light reflected from the bullseye of the second target exceeds an intensity threshold; a calculation of a percentage score by dividing the length value of the subset time period by the first time period; and performing at least one action selected and reporting the percentage score to a display device.

12. The illuminable orthostatic reporting system of claim 9 further comprising a system comprising: a processor; a light sensor communicatively coupled to the processor; and a non-transitory storage memory storing instructions that when executed by the processor cause the processor to perform operations, the operations comprising: a receiving during a first time period, via the light sensor, light intensity data corresponding to an amount of light reflected from the bullseye of the second target; a determination of a length value of a subset time period of the first time period for which the light intensity data corresponding to the amount of light reflected from the bullseye of the second target exceeds an intensity threshold; a calculation of a percentage score by dividing the length value of the subset time period by the first time period; and a reporting of the percentage score to a display device.

13. The illuminable orthostatic reporting system of claim 9 further comprising a system comprising: a processor; a light sensor communicatively coupled to a processor and coupled to a light sensor that is not physically coupled to the target; the non-transitory storage medium storing a set of instructions configured to instruct the processor to provide biofeedback to the user and display on the display device.

14. A method for a user to hold a desirable posture, the method comprising: providing a target having a bullseye; securing an illuminable orthostatic reporting apparatus, comprising an adjustable housing assembly and a laser pointer, to the user; maneuvering the user to a distance from the target; and adjusting the adjustable housing assembly such that a laser beam, having an angle with respect to the horizontal plane, being emitted from the laser pointer is projected onto the bullseye of the target simultaneously with the user having the desirable posture.

15. The method of claim 15 wherein the user maintains the desirable posture while simultaneously performing an exercise and maintaining the adjustable housing assembly at a substantially constant angle so as to cause the laser beam to constantly project, for at least 0.1 seconds, onto the bullseye of the target.

16. An orthostatic reporting system comprising: a forefoot pressure monitor wirelessly connectable to a system comprising a non-transitory medium and processor; and
a heel pressure monitor wirelessly connectable to a system comprising a non-transitory medium and processor.

17. An orthostatic reporting system comprising: a chest angle sensor coupled to the orthostatic reporting assembly; a chin angle sensor which may be held to the chin by straps around the ears; and a head crown angle sensor.

18. An orthostatic reporting system comprising: an angle capturing wireless connection accessible non-transitory storage medium connected camera configured to monitor body angle sensors and transmit findings to a non-transitory storage medium.

19. The system of claim 17, wherein the wireless connection accessible system comprises one or more of: a prompt sound responsive biometric feedback; and a data collection and processing response feature.

20. The system of claim 17, wherein the user reacts to biometric feedback while performing the method of claim 15 and thereby creating data for the system of claim 17 to process.
